(19) United States
(12) Patent Application Publication
(10) Pub. No.: US 2022/0074739 A1
(43) Pub. Date: Mar. 10, 2022

(54) MEASURING SYSTEM FOR MEASURING ARTICLES OF CLOTHING

(71) Applicant: Sascha DROSTE, Gronau (DE)

(72) Inventor: Sascha DROSTE, Gronau (DE)

(21) Appl. No.: 17/416,270

(22) PCT Filed: Dec. 17, 2019
(86) PCT No.: PCT/EP2019/085587

§ 371 Date: Jun. 18, 2021

(30) Foreign Application Priority Data
Dec. 20, 2018 (DE) 10 2018 133 045.6

(51) Int. Cl. G01B 11/25; G01N 33/36

(52) U.S. Cl. CPC G01B 11/2518; G01N 33/367

(57) ABSTRACT

A measuring system for measuring articles of clothing, comprising:
a measuring table which has a table surface on which the article of clothing to be measured is laid;
at least one laser distance meter which emits a laser beam, the laser distance meter being arranged in such a way that the laser beam extends in parallel with the table surface 4 at a short distance so that, when an article of clothing lies on the measuring table, the laser beam hits the article of clothing;
at least one marking on which the article of clothing is laid before a measurement is carried out by means of the at least one laser distance meter; and
a data-processing device which determines a length dimension of the article of clothing using the position of the at least one marking and using a value measured by the one or more laser distance meters.

The invention relates to a measuring system for measuring articles of clothing, comprising a measuring table having a table surface, the articles of clothing being placed onto said table surface and then being measured.

### BACKGROUND OF THE INVENTION

In the clothing industry it is routine practice to inspect the articles of clothing with regard to the quality and accuracy of the dimensions after production. For this purpose, the articles of clothing are placed onto a measuring table and visually inspected by an inspector. Moreover, various dimensions such as, for example, the waist measurement, hip measurement or the inseam length of pants are checked by hand using a measuring tape. The individual measurement data are noted and can then be compared with the target values predefined for the article of clothing. However, such a manual procedure is relatively complicated and time-consuming.

### OBJECT OF THE INVENTION

It is therefore an object of the present invention to provide a measuring system for measuring articles of clothing which makes the entire measuring procedure easier and faster.

This object is achieved according to the invention by means of the features mentioned in independent claim 1. Further embodiments of the invention are evident from the dependent claims.

The invention proposes a measuring system for measuring articles of clothing, comprising at least the following components:
a measuring table having a table surface, the article of clothing to be measured being placed onto said table surface,
at least one laser distance measuring device which emits a laser beam, wherein the laser distance measuring device is arranged such that the laser beam passes parallel to the table surface at a small distance so that, when an article of clothing is on the measuring table, said laser beam impinges laterally on the article of clothing,
at least one marking on which the article of clothing is placed before a measurement is carried out by means of the at least one laser distance measuring device, and
a data processing device, which determines a certain length dimension of the article of clothing taking account of the position of the at least one marking and a value measured by the one or more laser distance measuring devices. This has the advantage that the length measurement can be carried out significantly faster and more easily.

The data processing device can be a conventional PC, for example. By way of example, an analog sensor of the HG-C series from Panasonic can be used as laser distance measuring device.

In accordance with one preferred embodiment of the invention, the measuring system comprises a plurality of laser distance measuring devices. In this case, preferably at least two of the laser distance measuring devices are arranged on different sides of the article of clothing or of a region of the table surface onto which the article of clothing to be measured is placed.

The at least one marking mentioned above is preferably generated by means of a light source, in particular by means of a laser light source. The light source is preferably arranged above the measuring table and projects the marking, such as e.g. a point of light, onto the measuring table or the article of clothing. Alternatively, however, a marking, such as e.g. a colored point or line, can also be applied to the table surface.

A plurality of auxiliary lines can also be arranged on the table surface and assist an inspector when orienting the articles of clothing to be measured on the table surface.

The one or more laser distance measuring devices is/are preferably mounted on the table surface of the measuring table.

In accordance with one preferred embodiment of the invention the measuring system furthermore comprises a scanner, by means of which the article of clothing to be measured can be scanned in. In this case, the term “scan in” should be understood to mean that information, such as e.g. a barcode or other indications contained on a label, is read in and can be used to determine the target dimensions of the relevant article of clothing. As a result, it becomes possible to compare the actual values with the target values of the specific article of clothing.

The measuring system according to the invention can furthermore comprises a screen, on which the measurement result is displayed. In addition, the target dimensions of the article of clothing or a deviation between the actual and target values can also be displayed.

In one embodiment comprising a plurality of laser distance measuring devices, preferably at least two of the laser distance measuring devices are arranged at an angle with respect to one another, such that the laser beams emitted by the two laser distance measuring devices intersect in a region of the table surface onto which the article of clothing to be measured is placed (at least if said laser beams pass at the same height above the table). By way of example, the hip measurement of pants can be measured well by means of the laser distance measuring devices arranged at an angle.

In accordance with one preferred embodiment of the invention, the measuring system comprises exactly three laser distance measuring devices.

### BRIEF DESCRIPTION OF THE DRAWINGS

The invention is explained in greater detail by way of example below with reference to the accompanying drawings, in which:

FIG. 1 shows a plan view of a measuring system for measuring articles of clothing in accordance with one embodiment of the invention;

FIG. 2 shows a plan view of the measuring system from FIG. 1, wherein the measuring system is used for measuring the waist measurement of pants;

FIG. 3 shows a plan view of the measuring system from FIG. 1, wherein the measuring system is used for measuring the hip measurement of pants; and

FIG. 4 shows a plan view of the measuring system from FIG. 1, wherein the measuring system is used for measuring the inseam length of pants.

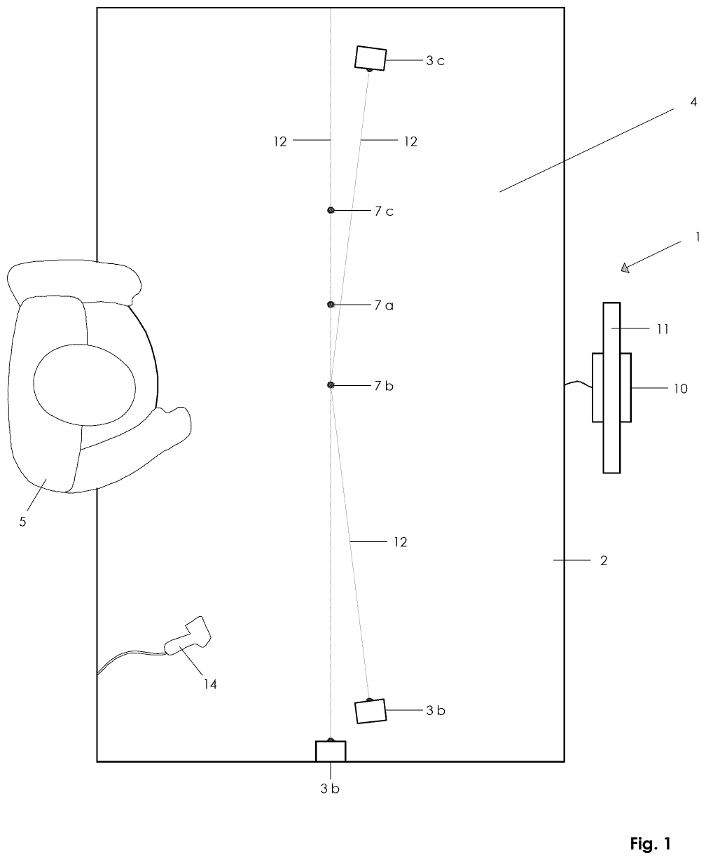

FIG. 1 shows a measuring system 1 for measuring articles of clothing 6, which enables an inspector to check the dimensions of a produced article of clothing 6. The measuring system 1 comprises a measuring table 2 having a table surface 4, the article of clothing 6 to be checked being placed onto said table surface. A plurality of—here three—laser distance measuring devices 3a-3c are arranged on the table surface 4 and each emit a laser beam 8a-8c that passes parallel to the table surface 4 at a small distance (e.g. 0.3 cm to 0.8 cm) so that, when an article of clothing 6 is arranged on the measuring table 2, said laser beam impinges on the article of clothing 6 on the side.

The individual laser distance measuring devices 3a-3c are arranged on the table surface 4 of the measuring table 2 such that various measurements of an article of clothing such as, for example, the waist measurement, the hip measurement or the inseam length can be measured accurately. For measuring straight dimensions such as, for example, the waist measurement or the inseam length of pants, only a single laser distance measuring device—here the laser distance measuring device 3a—is required. For measuring the hip measurement, the laser distance measuring devices 3b and 3c are provided here. The last two laser distance measuring devices 3b, 3c mentioned are arranged at an angle with respect to one another and each emit a laser beam 8b, 8c, which intersect in a region of the table surface 4 onto which the article of clothing 6 to be checked is placed.

Depending on the type of measurement, one or more of the laser distance measuring devices 3a-3c are activated. The one or more laser distance measuring devices 3a-3c not required is/are then preferably deactivated.

Furthermore, a plurality of auxiliary lines 12 are arranged on the table surface 4 and help the inspector in correctly orienting the articles of clothing 6 to be checked.

Moreover, a plurality of markings 7a-7c are discernible on the table surface 4 of the measuring table 2 in FIG. 1, which markings serve as a reference point for the inspector 5 and indicate to the latter where the article of clothing 6 must be placed before a laser measurement is carried out. Three markings 7a-7c serving as placement points for different measurements are provided in the exemplary embodiment illustrated.

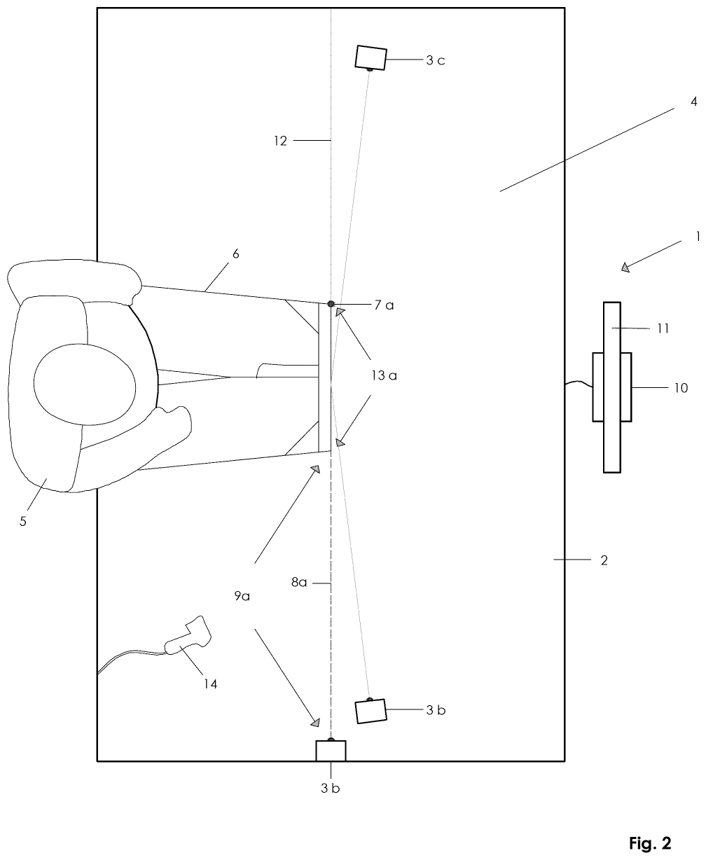

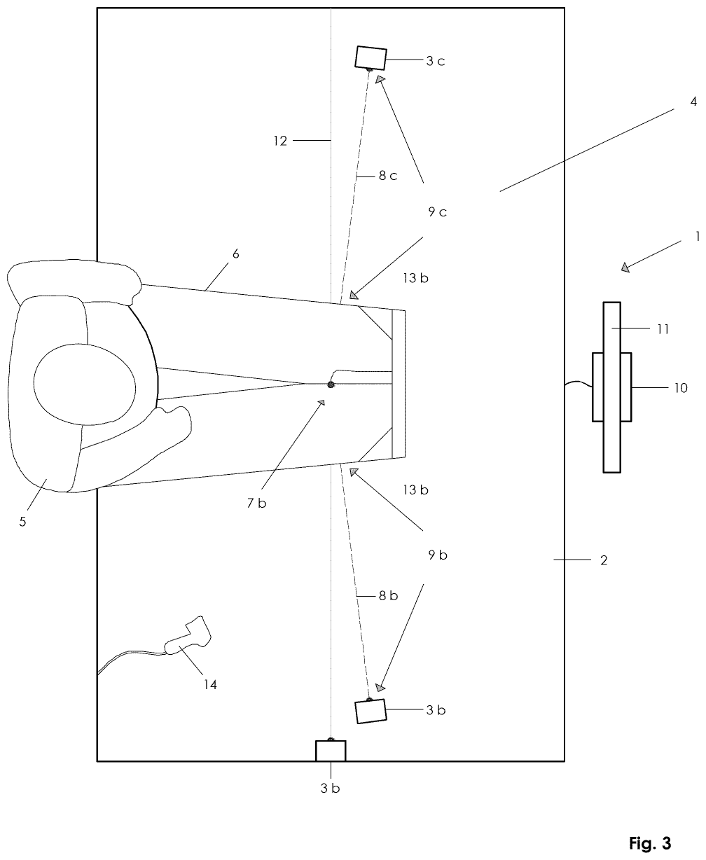

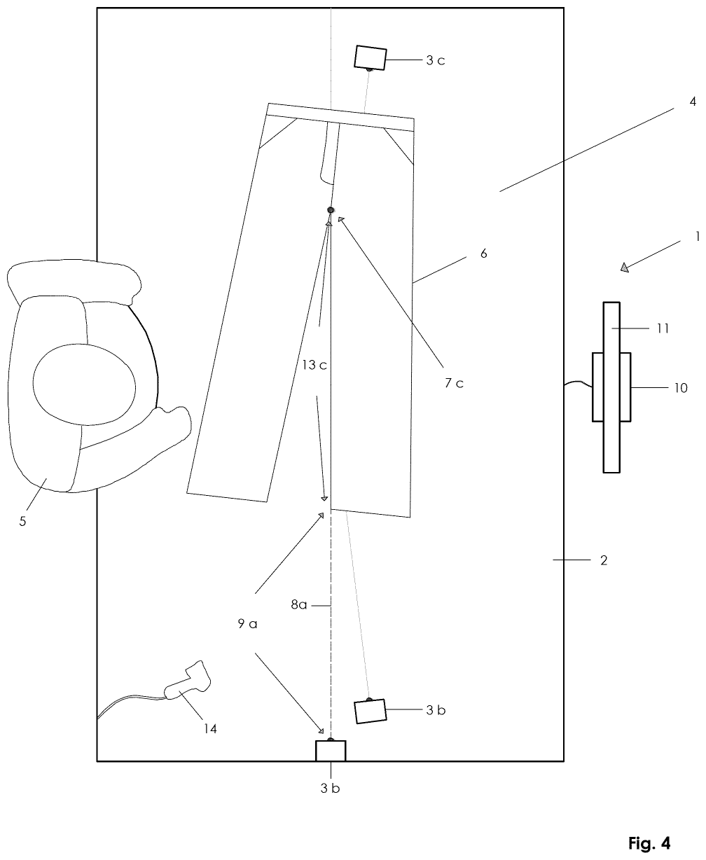

In the exemplary embodiment illustrated, the marking 7a marks a placement point at which the inspector 5 must place for example the waist of pants laterally in order to measure the waist measurement 13a, as is shown in FIG. 2. The marking 7b serves to indicate to the inspector 5 where the latter must place for example a cross seam of pants in order to measure the hip measurement 13b of pants, as is illustrated in FIG. 3, and the marking 7c serves to indicate to the inspector 5 where the latter must place the cross seam of pants in order to measure for example the inseam length of pants, as is illustrated in FIG. 4.

The individual markings 7a-7c are preferably generated by a respective light source (not shown), in particular a laser light source, which is arranged above the measuring table 2. Depending on the type of measurement, preferably only one of the markings 7a-7c is visible. The other light sources are preferably inactive.

The measuring system illustrating in FIG. 1 preferably also comprises a scanner 14, by means of which the article of clothing 6 to be checked can be scanned. The scanner used can be for example a barcode laser or some other optical scanner which is able to recognize a code or other optical features of an article of clothing 6 to be checked and is able to determine therefrom the associated target dimensions of the relevant article of clothing 6 such as e.g. a target waist measurement, a target hip measurement or a target inseam length. In accordance with one specific embodiment of the invention, a scanner 14 can be provided, for example, which is able to read a label attached to the article of clothing 6 and to recognize thereon for example the name of the manufacturer and a size indication by means of character recognition.

The measuring system furthermore comprises a data processing device 10, which is connected to the laser distance measuring devices 3a-3c, the scanner 14 and/or the light sources (not shown) and drives the individual components 3a-3c, 14 and also processes the data supplied by the components 3a-3c, 14. Moreover, a screen 11 is provided, on which the respective measurement result is displayed.

The functioning of the measuring system illustrated in FIG. 1 is explained in greater detail below on the basis of various examples.

FIG. 2 shows the measuring system 1 from FIG. 1, wherein the waist measurement 13a of pants is measured. In a first step, the inspector 5 firstly scans in the pants with the aid of the scanner 14. The scanned data are then communicated to the data processing device 10, which in turn determines therefrom the target dimensions of the article of clothing 6, in the present case for example the target waist measurement, the target hip measurement and the target inseam length of the pants to be checked. The target values are stored in a database.

Since the waist measurement 13a of the pants is being measured in the present case, only the marking 7a is displayed, this being the marking that marks a placement point of the waist of the pants. The inspector 5 can then place the pants on the marking 7a in the prescribed manner (as illustrated). The auxiliary lines 12 indicated on the tale surface 4 assist the inspector here when orienting the pants on the measuring table 2.

As soon as the pants are in the correct position, the inspector 5 presses a foot pedal or a button (not shown), as a result of which the waist measurement is measured by means of the laser distance measuring device 3a. In this case, the laser distance measuring device 3a measures a measurement distance 9a between the laser distance measuring device 3a and the waist of the pants. Since the position of the marking 7a is known, the waist measurement 13a can be determined taking account of the position of the marking 7a and the measurement distance 9a. The waist measurement 13a is preferably determined in the data processing device 10. The measurement result and optionally also a deviation from the target waist measurement can optionally be displayed on a screen 11. The measurement result is preferably stored and can be retrieved again at a later point in time.

When the waist measurement 13a is being measured, preferably only the laser distance measuring device 3a is active. The other two laser distance measuring devices 3b, 3c are preferably deactivated.

FIG. 3 shows a view of the measuring system 1 from FIG. 1, which measuring system in this case is used to measure the hip measurement 13b of pants.

When the hip measurement is being measured, only the marking 7b is projected onto the measurement table 2. The other two light sources are preferably switched off. The marking 7b indicates to the inspector 5 where the latter ought to place a cross seam of the pants. The auxiliary lines 12 once again assist the inspector when orienting the pants.

Pressing of the foot pedal (not shown) once again initiates a measurement—but this time by means of the two laser distance measuring devices 3b and 3c. The laser distance measuring device 3a is switched off. Each of the laser distance measuring devices 3b, 3c measures a respective measurement distance 9b, 9c between the relevant distance measuring device and the hip of the pants. Since the position of the marking 7b is in turn known, the hip measurement 13b of the pants can be calculated taking account of this position and the two measurement distances 9b, 9c. The hip measurement 13b is preferably determined in the data processing device 10. The measurement result and optionally a deviation from the target hip measurement can be displayed for example on a screen 11.

Finally, FIG. 4 also shows the measuring system 1 from FIG. 1, which measuring system in this case is used for measuring the inseam length 13c of pants. When the inseam length has been measured, only the marking 7c is indicated. The other two light sources are switched off. The inspector 5 can then place the pants on the marking 7c in the predefined manner and orient the pant leg to be checked on the auxiliary line 12, as is illustrated in FIG. 4.

In order to carry out the measurement, the inspector 5 once again presses the foot pedal. In this case, the measurement is carried out exclusively by the laser distance measuring device 3a, which measures a measurement distance 9a between the laser distance measuring device 3a and the end of the pant leg. Since the position of the marking 7c and the measurement distance 9a are then known, the inseam length 13c of the pants can in turn be calculated taking account of these values.

The measuring system is preferably set up in such a way that after each measurement the appropriate configuration for the subsequent measurement is set automatically. After the waist measurement has been measured, for example, the measuring system 1 is automatically configured such that the hip measurement can be measured in a next measurement, and then the inseam length of pants can be measured in a subsequent measurement. In this context, for example, the system defines which laser distance measuring device 3a-3c is switched in active or passive fashion, which light source is selected for generating a marking 7a-7c or which data are transferred to the data processing device and processed.

The number, type and order of the individual measurements can be defined as desired, in principle. The entire measuring procedure is preferably controlled by means of the data processing device 10. However, some other control unit can also be provided.

1. A measuring system for measuring articles of clothing, comprising:
a measuring table having a table surface, the article of clothing to be measured being placed onto said table surface,
at least one laser distance measuring device which emits a laser beam, wherein the laser distance measuring device is arranged such that the laser beam passes parallel to the table surface at a small distance so that, when an article of clothing is on the measuring table, said laser beam impinges on the article of clothing,
at least one marking on which the article of clothing is placed before a measurement is carried out by means of the at least one laser distance measuring device, and
a data processing device, which determines a length dimension of the article of clothing taking account of a value measured by the one or more laser distance measuring devices.

2. The measuring system as claimed in claim 1, wherein the data processing device takes account of the position of the at least one marking when determining the length dimension.

3. The measuring system as claimed in claim 1, wherein the at least one laser distance measuring device comprises a plurality of laser distance measuring devices.

4. The measuring system as claimed in claim 3, wherein at least two of the laser distance measuring devices are arranged on different sides of a region of the table surface onto which the article of clothing to be measured is placed.

5. The measuring system as claimed in claim 1, wherein the at least one marking is generated by means of a light source, in particular by means of a laser light source, which is arranged above the measuring table.

6. The measuring system as claimed in claim 1, wherein a plurality of auxiliary lines are arranged on the table surface and assist an inspector when orienting the articles of clothing on the table surface.

7. The measuring system as claimed in claimed in claim 1, wherein the at least one laser distance measuring device is mounted on the table surface of the measuring table.

8. The measuring system as claimed in claim 1, further comprising a screen, on which the measurement result of the measurement carried out by means of the at least one laser distance measuring device is displayed.

9. The measuring system as claimed in claimed in claim 1, it wherein the at least one laser distance measuring device comprises a plurality of laser distance measuring devices, wherein at least two of the laser distance measuring devices are arranged at an angle with respect to one another, such that the laser beams emitted by the two laser distance measuring devices intersect in a region of the table surface onto which the article of clothing to be measured is placed.

10. The measuring system as claimed in claimed in claim 1, wherein the at least one laser distance measuring device comprises exactly three laser distance measuring devices.
